(19) United States
(12) Patent Application Publication
(10) Pub. No.: US 2023/0158490 A1
(43) Pub. Date: May 25, 2023

(54) DEVICE

(71) Applicants: Volker Martin Lauschke, Stockholm (SE); Reza Zandi Shafagh, Täby (SE); Wouter van der Wijngaart, Sollentuna (SE)

(72) Inventors: Volker Martin Lauschke, Stockholm (SE); Reza Zandi Shafagh, Täby (SE); Wouter van der Wijngaart, Sollentuna (SE)

(21) Appl. No.: 17/920,154

(22) PCT Filed: Apr. 23, 2021
(86) PCT No.: PCT/SE2021/050379

§ 371 Date: Oct. 20, 2022

(30) Foreign Application Priority Data
Apr. 24, 2020 (GB) 2006067.9

(51) Int. Cl. B01L 3/00; C12M 3/06

(52) U.S. Cl. CPC B01L 3/502707; B01L 3/50273; B01L 3/502738; B01L 3/502715; B01L 3/5085; C12M 23/16; B01L 2200/0689; B01L 2400/0487; B01L 2300/0861; B01L 2300/14; B01L 2300/0848

(57) ABSTRACT

The present invention is directed towards a microfluidic device comprising a first compartment comprising an inlet that is connectable to a fluidic control unit and a second compartment, wherein the first and second compartments are connected by a micrometer channel so as to allow fluid communication between the two compartments. The device also comprises an air-lock element in fluid communication with the second compartment and the air-lock element is configured so that in use the internal atmosphere of the device is sealed from the external atmosphere and so that when fluid is introduced or withdrawn from the first compartment via the inlet the air-lock element maintains an overall constant pressure within the device.

The present invention is also directed towards a method of manufacturing the microfluidic device, a kit-of-parts comprising the microfluidic device and a method of using the microfluidic device for accommodating, growing, culturing, isolating, treating and/or processing cells.

The present invention is directed towards a microfluidic device comprising a first compartment comprising an inlet that is connectable to a fluidic control unit and a second compartment, wherein the first and second compartments are connected by a micrometer channel so as to allow fluid communication between the two compartments. The device also comprises an air-lock element in fluid communication with the second compartment and the air-lock element is configured so that in use the internal atmosphere of the device is sealed from the external atmosphere and so that when fluid is introduced or withdrawn from the first compartment via the inlet the air-lock element maintains an overall constant pressure within the device.

The present invention is also directed towards a method of manufacturing the microfluidic device, a kit-of-parts comprising the microfluidic device and a method of using the microfluidic device for accommodating, growing, culturing, isolating, treating and/or processing cells.

Tissue interactions using microfluidic devices have been studied before. Examples for co-culture devices include microfluidic liver-lung (Viravaidya et al. Biotechnol Progr 2008; 20(1): 316-323), fat-kidney-liver-lung (Zhang et al. Lab Chip 2009; 9(22): 3185-3189), gut-skin-liver-kidney (Maschmeyer et al. Lab Chip 2015; 15: 2688-2699) and heart-muscle-brain-liver (Oleaga et al. Sci Rep 2016; 6: 20030) systems and these efforts have been successfully applied in pharmacokinetic modeling (Kim et al. Lab Chip 2012; 12(12): 2165-2110; Sung et al. Lab Chip 2009; 9(10): 1385-1310; Imura et al. Analyt Chem 2013; 85(3): 1683-1688), analysis of disease mechanisms (Song et al. PLoS ONE 2009; 4(6): e5756-5710; Kim et al. PNAS 2016; 113(1): E7-15), as well as evaluations of drug efficacy (Aref et al. Integr Biol 2013; 5(2): 381-389; Tatosian et al. Biotech Bioeng 2009; 103(1): 187-198; Vidi et al. Lab Chip 2014; 14(1): 172-177) and toxicity (Choucha et al. Biotech Bioeng 2013; 110(2):597-608). Importantly however, current platforms consist primarily of immortalized cell lines or tissue-specifically differentiated stem cells (Rogal et al. Adv Drug Delivery Rev 2019; 140:101-128), and the only multi-tissue platforms for studies of T2D presented to date integrates pancreatic islets with hepatoma cells (Bauer et al. Sci Rep 2017; 7(1): 14620). Furthermore, most models are cultured in 2D monolayers supported by various matrices or scaffolds, such as matrigel (Trietsch et al. Lab Chip 2013; 13(18): 3548-3548), polyethyleneglycol (Mao et al. Lab Chip 2012; 12(1): 219-226), polylactic acid (Ma et al. Biomaterials 2012; 33(17): 4353-4361) or alginate (Sung et al. Lab Chip 2009; 9(10): 1385-1310), which interfere with drug absorption, increase batch-to-batch variability and further compromise the physiological relevance of these models.

However, significant improvements remain for improved tissue modeling microfluidic devices and the claimed invention provides these improvements.

In particular, there are at least five surprising advantages to the claimed invention:
1) actuating the liquid in the first (central) compartment suffices to control the liquid in all other compartments thereby enabling reciprocal cross-talk between the cells or tissue models cultured in the individual compartments;
2) molecular signals, be they metabolites, proteins, nucleic acids or ions, from the individual radial/satellite compartments are integrated in the central compartment from where they are then distributed back out to the other compartment(s) connected to this said first (central) compartment;
3) the stoichiometry of flows to the individual compartments can be controlled;
4) the flow in each of the compartments may be periodically alternated in direction (influx to and efflux from the compartment(s));
5) the use of one or more air-lock elements prevents pressure build-up while strongly limiting evaporation from cell culture medium within the device and the risk of contamination from the outside environment.

### BRIEF DESCRIPTION OF THE INVENTION

According to the present invention, there is provided a microfluidic device comprising:
a first compartment comprising an inlet that is connectable to a fluidic control unit;
a second compartment;
a micrometer channel connecting the first and second compartments so as to allow fluid communication between the first and second compartments; and
an air-lock element in fluid communication with the second compartment, wherein the air-lock element is configured so that in use the internal atmosphere of the device is sealed from the external atmosphere and so that when gas is introduced or withdrawn from the first compartment via the inlet the air-lock element maintains an overall constant pressure within the device.

According to another aspect of the invention, there is provided method of manufacturing a device as defined above.

According to a further aspect of the invention, there is provided a method of using a device as defined above for accommodating, growing, isolating, treating and/or processing cells.

According to another aspect of the invention, there is provided a kit-of-parts comprising a microfluidic device according to the invention in the form of a sterile, pre-packaged kit-of-parts for single use.

The device of the invention is intended for use as a microfluidic device that is particularly suited for growing different cell types in at least one of the compartments and allowing fine control of the flow of the liquid (e.g. cell culture medium) around the device by modulating the gas pressure in the upper section of the first compartment via the inlet. Through periodically increasing and decreasing the gaseous pressure in the first compartment, flow of liquid medium between the compartments can be modulated allowing mixing of the medium that mimics the mixing of bodily fluids from different bodily organs. Therefore, the device of the invention is particularly suited for modeling bodily systems and how different organs interact through fluid exchange in the body.

### DEFINITIONS

In general, the term “micrometer channel” followed by an “s” in parenthesis (i.e. micrometer channel(s)) intends to cover all embodiments where the device comprises only a single micrometer channel as well as those embodiments where the device comprises a plurality of micrometer channels, and the particular optional feature being discussed may be comprised by only one of the channel(s) in question or in a multiple of channels, such as all of the channels. Unless defined to the contrary in specific embodiments, the term “micrometer channel(s)” refers to the lower channels connecting the compartments of the device together and allowing liquid fluid communication therethrough, e.g. the micrometer channels that the cell culture medium flows through when in use.

The term “compartment” as used herein refers to a void or well within the device in which cells may be contained. Such compartments comprise a bottom floor section and a wall section, which is interrupted by the presence of the opening to the micrometer channel(s). These compartments may also be referred to interchangeably herein as “cell compartments”. However, although all compartments in the device may be suitable for accommodating and growing cells, in use it does not necessarily mean that all compartments are used as such. For example, when in use in some embodiments the first (central) compartment may be absent of cells within it and the first (central) compartment may act as a compartment for mixing the cell culture medium as it flows from the radial/satellite compartments into the first compartment to then be redistributed out to these compartments.

Depending on the number of compartments within the device, the “first compartment” of the device is also referred to herein interchangeably as the “central compartment”.

In use the device is under standard pressure relative to the environment external to the device. On the introduction or retraction of gas into or out of the upper section of the first compartment the pressure within the device fluctuates by no more than about 20%, such as no more than about 10%, for example no more than about 5%, 4%, 3%, 2% or 1% in relation to the standard pressure. This definition is encompassed herein under the term “overall constant pressure”.

### BRIEF DESCRIPTION OF THE DRAWINGS

The embodiments of the invention, together with its advantages, may be best understood from the following detailed description taken in conjunction with the accompanying figures.

FIG. 1 is a schematic of a microfluidic device according to the invention (with the air-lock element not shown) that comprises two compartments.

FIG. 2 is a top view of a microfluidic device according to the invention (with the air-lock element not shown) that comprises five compartments.

FIG. 3 is a top side view of a microfluidic device according to the invention (with the air-lock element not shown) that comprises five compartments.

FIG. 4a is a top side view of a microfluidic device according to the invention that comprises five compartments and one oil-lock system.

FIG. 4b is a top view of a microfluidic device according to the invention that comprises five compartments and an oil-lock system for each satellite compartment.

FIGS. 5a to 5f depict a side view of a microfluidic device according to the invention that is in use.

FIG. 7 is a plot showing the long-term stability of primary humans cells in a device according to the invention compared to a standard.

FIG. 8 is a plot showing change in expression levels as a function of glucose concentrations for insulin responsive genes in the liver compartment of co-culture chips with human liver spheroids and pancreatic islets.

### DETAILED DESCRIPTION OF THE INVENTION

The invention should not be construed as being limited to any of the following embodiments or any of the features described in these embodiments except to those features present in the independent claims. Furthermore, it is envisaged that all features in the embodiments may be combined with other features in other embodiments where appropriate and reasonably plausible.

The Microfluidic Device

According to the present invention, a microfluidic device comprising:
a first compartment comprising an inlet that is connectable to a fluidic control unit;
a second compartment;
a micrometer channel connecting the first and second compartments so as to allow fluid communication between the first and second compartments; and
an air-lock element in fluid communication with the second compartment. The air-lock element is configured so that in use the internal atmosphere of the device is sealed from the external atmosphere and so that when fluid is introduced or withdrawn from the first compartment via the inlet the air-lock element maintains an overall constant pressure within the device.

Advantageously, the fluidic control unit is a gaseous fluid control unit, Conveniently, the fluid introduced or withdrawn from the first compartment via the inlet is a gas.

Preferably, the micrometer channel connecting the first and second compartments allows liquid fluid communication.

By the term “air-lock element in fluid communication with the second compartment” it is envisaged that this communication may be direct (i.e. the second compartment is directly connected to the air-lock element) or it may be indirect (i.e. the connection may be via a further compartment, e.g. a further radial/satellite compartment).

Advantageously, the air-lock element is in the form of a liquid air-lock. That is to say, the liquid air-lock comprises a section that contains liquid, wherein the internal atmosphere of the device and the atmosphere external to the device is separated by the liquid in the liquid air-lock.

Preferably, the air-lock element is a liquid air-lock that comprises a first chamber and a second chamber connected via a lower channel to allow fluid communication therethrough and wherein the first chamber is connected to the second compartment, either directly or indirectly, via an upper channel to allow fluid communication therethrough. It is envisaged that the first and second chamber, along with the connecting lower channel, can be distinct (e.g., at right angles to one another) or they can be smoothly integrated to form, for example, a U-bend.

Preferably, the liquid used in the liquid air-lock is selected from the list consisting of mineral oils (e.g. paraffin oil) and vegetable oils.

Advantageously, the second chamber of the air-lock element comprises an outlet to allow gas to flow into and out of the upper section of the second chamber.

Conveniently, the second chamber comprises a base, wherein the outlet is positioned in the device at a location further from the base of the second chamber than the lower channel.

In use, a pressure control unit may be connected to the inlet of the first compartment and the pressure control unit may be configured to modulate the gas pressure in the upper section of the first compartment by the introduction or retraction of a gaseous medium into or out of the first compartment so as to control fluid flow between the compartments via the micrometer channel.

Advantageously, the pressure control unit is a high-precision pump or a syringe pump. Conveniently, the syringe pump is configured to pump and withdraw at the syringe flow rate of from about 1 to about 10 mL/min, such as about 1 to about 5 mL/min, for example about 2 to about 4 mL/min.

When in use with a pressure control unit, as the gaseous pressure increases in the first compartment, the liquid in the first compartment drops in level and flows through the micrometer channel into the second compartment. As a result, the liquid level in the second compartment increases, thus increasing the gaseous pressure in the upper section of the second compartment. The increase in pressure in the upper section of the second compartment is regulated by the air-lock element, which preferably contains a liquid that is displaced when the gaseous pressure in the upper section of the second compartment increases. Therefore, this configuration allows the flow of the liquid within the device to be controlled in use whilst not putting the device under pressure and simultaneously prohibiting exposure of the cells growing in the device to the external environment.

Preferably, the inlet of the first compartment is positioned in the device at a location above the level of the micrometer channel. More preferably, the inlet is positioned directly above the first compartment. Put another way, the compartments comprise a floor section and a wall section which define the compartment, in this embodiment the inlet is positioned directly above and opposite to the floor section of the first compartment.

Advantageously, the device comprises a removable stopper or cover for closing the inlet.

Preferably, the first compartment comprises a base (also referred to herein as a floor section), wherein the inlet is positioned in the device at a location further from the base of the first compartment than the micrometer channel. This configuration results in the inlet being positioned in the device at a location above the level of the micrometer channel.

Advantageously, the first compartment comprises a wall section which, together with the base/floor section, defines the compartment. Preferably, all compartments of the device comprise a base/floor section and a wall section, which together define the compartments.

Conveniently, the inlet is positioned directly above and opposite to the floor section of the first compartment.

In use, the microfluidic device comprises a liquid, such as a cell culture medium and the device is filled so that a residual pocket of gaseous medium is present in the first compartment. The inlet is positioned above the line of the liquid so that the pressure control unit directly introduces gaseous medium into, or retracts gaseous medium out of, the pocket of gaseous medium present in the upper section of the first compartment.

When retracting gaseous medium out of the first compartment, this causes the liquid in the microfluidic device to flow towards and into the first compartment through the micrometer channel thus increasing the volume of liquid in the first compartment and lowering the volume of liquid in the second compartment.

Conversely, when gaseous medium is introduced into the pocket of gaseous medium in the first compartment, this causes the liquid in the microfluidic device to flow away from the first compartment and into the second compartment via the micrometer channel.

Advantageously, the microfluidic device comprises a further compartment (i.e. a third compartment) in direct fluid communication with the first compartment via a micrometer channel.

Conveniently, the microfluidic device comprises a plurality of further compartments, each of which are in direct fluid communication with the first compartment via micrometer channels. For example, the microfluidic device may comprise 3, 4, 5, 6, 7, 8, 9 or 10 compartments all in direct fluid communication with the first compartment. In this sense, the first compartment may also be referred to as a “central compartment” and the plurality of further compartments may be referred to as “radial compartments” or “satellite compartments”. In all embodiments where the microfluidic device comprises a first (central) compartment and a plurality of radial/satellite compartments, all of the radial/satellite compartments are in fluid communication, either directly or indirectly, with the first (central) compartment via micrometer channels as defined herein. In this sense, by direct communication it is envisaged that each of the radial/satellite compartments has a micrometer channel that directly leads to the first (central) compartment. By indirect fluid communication it is envisaged that the flow from the radial/satellite compartment may be via further elements of the device, such as through further radial/satellite compartments.

It is envisaged herein that where the device comprises three or more compartments, the second compartment as defined in the statement of invention may also be collectively referred to as part of the radial/satellite compartment array.

In this embodiment where the microfluidic device comprises a first (central) compartment and a plurality of radial compartments, in use the flow of the liquid medium in the device is controlled by a pressure control unit modulating the volume of gaseous medium in the first compartment. When introducing further gaseous medium into the first (central) compartment the liquid medium in the device is pushed towards and into the radial compartments via the micrometer channels. When retracting further gaseous medium into the first (central) compartment, the liquid medium is drawn from the radial compartments and into the first (central) compartment via the micrometer channels. This results in the medium from the radial compartments mixing in the first (central) compartment. Optionally, each of the radial compartments individually may be in fluid communication with any of the other radial compartments via (a) micrometer channel(s).

Preferably, each of the radial/satellite compartments are in fluid connection with each other via upper channels to allow gaseous exchange between the radial satellite compartments, wherein the upper channels of each of the compartments, either directly or indirectly, allow fluid communication therethrough with the first chamber of the air-lock element. Conveniently, the upper channels are not connected, either directly or indirectly, to the first compartment. Some currently used microfluidic devices modulate the flow therethrough using a membrane, or usually a series of membranes, in direct contact with the liquid medium which push and pull the medium around the device. In order to enable such devices to work, they must be completely full of liquid medium to be pushed through the device. However, in such instances the materials that comprise the devices, particularly the membranes, are not optimal for modeling in vivo processes and often cell clusters adhere to the surfaces of the membranes causing them to act in abnormal ways (such as undergoing unwanted differentiation etc.).

In the present inventive microfluidic device, this disadvantage is avoided as the mechanism of controlling fluid flow does not rely on membranes in direct contact with the fluid membrane.

Conveniently, the micrometer channel(s) has/have at least one dimension that is equal to or smaller than about 2000 μm, such as equal to or smaller than about 1500 μm.

Preferably, the micrometer channel(s) has/have, independently, a length of from about 0.1 to about 100 mm, for example from about 1 to about 100 mm, such as from about 5 to about 75 mm, for example from about 10 to about 50 mm, more preferably from about 15 to about 40 mm.

Advantageously, the micrometer channel(s) has/have a width (or a diameter when the micrometer channels are cylindrical, also referred to herein as hydraulic diameter), independently, of from about 1 to about 2000 μm, for example of from about 50 to about 2000 μm, such as from about 100 to about 1500 μm, for example from about 200 to about 1500 μm.

Conveniently, the micrometer channel(s) length(s) and/or width(s) are the same or different. That is to say, where it is stated that each of the micrometer channels “independently” have a dimension within the stated range, each of the micrometer channels may have any one of those dimensions and the dimensions may be the same with between all, or between some of, the micrometer channels or they may be different.

The micrometer channel(s) may be of any longitudinal shape that allows fluid flow therethrough. For example, the micrometer channels may be cylindrical or any other shape, such as a rectangular cuboid.

Preferably, the micrometer channel(s) may individually be essentially straight between the respective compartments, or the micrometer channel(s) may individually have a tortuous (i.e. meandering or serpentine) shape with at least one turn, preferably a plurality of turns.

The microfluidic device may be made of any suitable material as long as the internal faces of the compartments that are exposed to the cell cultures in use (i.e. the walls and floor) are compatible for cell growth. Advantageously, the internal walls of the compartments and/or the micrometer channels are comprised of a polymer selected from the list of poly(dimethyl siloxane) (PDMS); a thiol-ene polymer; a polymer with thiol, ene and/or epoxide groups on the surface; cyclic olefin copolymer (COC); polystyrene (PS); polycarbonate (PC); or an acrylic, such as poly(methyl methacrylate) (PMMA).

Conveniently, the floor surface of the compartments is comprised of the same material as the internal walls and/or micrometer channels, or the floor surface may comprised of a polymer with thiol and/or epoxide groups on the surface. For example, the floor surface of the compartments may be comprised of an off-stoichiometric thiol-ene polymer (OSTE) or an off-stoichiometry thiol-ene-epoxy (OSTE(+)).

OSTE and OSTE(+) polymers comprise off-stoichiometry blends of thiol and epoxide based monomer units. After polymerization, which can be typically carried out by UV micromolding, the resulting polymer article contains a number of unreacted thiol or epoxide groups both on the surface and in the bulk. These surface anchors can be used for subsequent direct surface modification or functionalisation without requiring surface activation. OSTE chemistry was first described in Carlborg, Lab Chip, 2011, Vol, 11(18), p. 3136-3147.

Advantageously, the internal faces of the compartments that are exposed to the cell cultures in use (i.e. the walls and floor) may be comprised OSTE or OSTE(+).

Preferably, the compartments each individually have an internal volume of from about to about 200 mm³, such as from about 5 to about 100 mm³, such as from about 10 to about 50 mm³, for example from about 15 to about 40 mm³.

The compartments may be of any shape as long as they are suitable for housing cells. Preferably, the compartments are cylindrical in shape, optionally where the compartments have an essentially flat bottom (i.e. a flat floor).

Preferably, the device comprises at least one aperture, such as an access port (e.g., fluid feed hole), located to allow input of a fluid into at least one of the compartments, preferably the first compartment. Advantageously, the aperture comprises a removable (i.e. reattachable) cover so as to allow the user to cover the aperture when not in use and prevent ingress of unwanted foreign materials into the wells. Conveniently, the aperture has a width ranging from 20 μm to 5000 μm.

Alternatively, or in addition to, the at least one aperture, one or more of the radial/satellite compartments comprise one or more capillary valves positioned in the compartment(s) to allow connection between the cell culture medium in use and the external atmosphere.

As used herein, the term “capillary valve” refers to passive non-mechanical valves which operate by surface tension provided by the cell culture medium within the capillary valve to block or restrict flow in the channel.

The incorporation of such a capillary valve(s) allows for the device to remain sealed to the external atmosphere whilst allowing for a sample to be taken from the cell culture medium via the channel, for example by using a syringe or a micropipette that is passed through the capillary valve. Such a valve does not require the use of a stopper to seal the valve, which reduces the chances of contaminating the cell culture medium.

Conveniently, the capillary valve(s) are positioned in the floor of the compartment(s) and extend through the bottom of the device to provide a connection between the cell culture medium in use and the external atmosphere.

Advantageously, the compartments of the microfluidic device are suitable for accommodating, growing, culturing, isolating, treating and/or processing cells. In particular, the device supports the use of cell lines, as well as primary cells of various tissue origin. For example, primary human hepatocytes, human cancer cell lines, as well as primary murine muscle bundles can be cultured in the device. The inventors have surprising found that these cells can successfully be cultured for multiple days without decreases in viability as judged by stable levels of intracellular ATP. As such, OSTE and OSTE(+) in general and the device design in particular are expected to also be compatible with cells from other sources.

Preferably, the microfluidic device comprises a bottom continuous layer, an upper layer and a sandwich layer disposed between the bottom continuous layer and the upper layer. The sandwich layer comprises at least two cut-outs extending through the plane of the sandwich layer and a micrometer channel connecting the two cut-outs. The layers of the device are positioned relative to one another so that the at least two cut-outs and the surface of the bottom continuous layer in contact with the sandwich layer define the first and second compartments of the microfluidic device.

By the term “continuous layer” it is meant a layer that does not comprise any apertures or holes so as to act as a seal layer as described below.

Depending on the number of compartments that the eventual device is intended to comprise, the sandwich layer will comprise the same number of cut-outs extending through the sandwich layer.

In an alternative embodiment, the bottom continuous layer and the sandwich layer may be unitary and the compartments may be prefabricated in this combined layer. In this embodiment, the air-lock element, for example the liquid air-lock element, may also be prefabricated within the continuous bottom and sandwich layer.

The bottom continuous layer may have a thickness of from about 100 μm to about 5000 μm, such as from about 200 μm to about 2000 μm, for example from about 300 μm to about 1000 μm.

The sandwich layer may have a thickness of from about 0.1 mm to about 100 mm, such as from about 0.2 mm to about 50 mm, for example from about 0.5 mm to about 10 mm.

The upper layer may have a thickness of from about 0.1 mm to about 100 mm, such as from about 0.2 mm to about 50 mm, for example from about 0.5 mm to about 10 mm.

Advantageously, the device further comprises a membrane layer disposed between the sandwich layer and the upper layer, optionally wherein the membrane layer is composed of poly(dimethyl siloxane).

The membrane layer may have a thickness of from about 10 μm to about 10 mm, such as from about 50 μm to about 7 mm, for example from about 100 μm to about 5 mm.

Once fully constructed the device may have a total thickness (i.e. height) of from about 0.5 mm to about 50 mm, such as from about 0.5 to about 30 mm, for example from about 1 mm to about 20 mm.

Conveniently, the sandwich layer and/or upper layer is/are comprised of a polymer selected from the list of poly(dimethyl siloxane) (PDMS), or an acrylic, such as poly(methyl methacrylate) (PMMA).

Preferably, the bottom continuous layer is comprised of the same material as the sandwich and/or upper layer. In a more preferable embodiment the bottom continuous layer is comprised of a polymer material with thiol and/epoxide groups on the surface of the layer disposed towards the sandwich layer, such as an OSTE or OSTE(+) polymer.

Unlike bottom layers comprised of commonly used materials, such as PDMS, the use of an OSTE or OSTE(+) based bottom layer results in a leak proof device which does not require an additional step, such as the application of adhesive, to attach to the sandwich layer.

Advantageously, in the embodiment where the bottom continuous layer is comprised of a polymer material with thiol and/or epoxide groups on the surface, this layer is attached to the sandwich layer by direct covalent bonding.

Advantageously, the upper layer and, if present, the membrane layer comprises an aperture extending therethrough and positioned above one of the compartments so as to define the inlet.

Method of Manufacturing the Microfluidic Device

Also according to the present invention there is provided a method of manufacturing a device as defined above.

The microfluidic device described herein is not limited to being formed by any particular manufacturing process.

The wells and/or micrometer channel(s) may be manufactured by etching, microfabrication methods (such as photolithography, soft lithography, and film deposition), or micromachining (such as laser micromachining) a substrate.

Preferably the microfluidic device comprises a bottom continuous layer, an upper layer and a sandwich layer disposed between the bottom continuous layer and the upper layer. The sandwich layer comprises at least two cut-outs extending through the plane of the sandwich layer and a micrometer channel connecting the two cut-outs. The layers of the device are positioned relative to one another so that the at least two cut-outs and the surface of the bottom continuous layer in contact with the sandwich layer define the first and second compartments of the microfluidic device.

Depending on the number of compartments that the eventual device is intended to comprise, the sandwich layer will comprise the same number of cut-outs extending through the sandwich layer.

In an alternative embodiment, the bottom continuous layer and the sandwich layer may be unitary and the compartments may be prefabricated in these layers. In this embodiment, the liquid air-lock element may also be prefabricated within the continuous bottom and sandwich layer.

Preferably, the bottom continuous layer, the sandwich layer and the upper layer are composed of an OSTE or OSTE(+) polymer. Such a device may be manufactured via the following manufacturing steps.

The fabrication process of OSTE+ microfluidic devices is implemented by OSTE(+) Micro Reaction Injection Molding (μRIM) process comprised of three main steps:

The OSTE or OSTE(+) polymer may be injected into a mold, preferably wherein the mold is a metal mold, such as an aluminium mold.

The mold may be configured to provide flat layers for subsequent construction, or where the bottom layer and sandwich layer are unitary the mold may comprise projections and cavities corresponding to the eventual compartments, micrometer channels and air-lock elements that the device will contain.

Preferably, after injection into the mold the polymer is then UV cured.

Advantageously, the polymer cast is then demolded out of mold, preferably either manually or by ejector pins.

In the embodiments where the device comprises a PDMS membrane layer between the sandwich layer and upper layer, the membrane layer may be fabricated via the casting of PDMS resin inside a cavity and subsequent thermal curing from about 10 to about 120 minutes, preferably from about 30 to about 60 minutes depending on the layer thickness.

The sandwich layer can be either directly micromachined for the purpose of flexible prototyping or alternatively μRIMed when the optimized feature sizes are established. The μRIM of the sandwich layer follows the steps as stated above.

Conveniently, all layers are assembled together in the arrangement as detailed above and undergo a second curing step. Preferably the second curing step is by exposure to heat at a temperature of from about 50° C. to about 150° C., such as from about 60° C. to about 100° C. for about 60 to about 180 minutes, such as about 100 to about 140 minutes. This step leads to strong covalent bonding of all layers together except the PDMS layer relies on conformal contact.

OSTE (+) polymer systems typically follow a dual curing procedure, in which the first curing step, typically achieved by UV curing, crosslinks the resin. Subsequently, the second curing step, typically achieved by thermal curing, hardens the already cured polymer.

Advantageously, the time window between first and second curing can be harnessed to implement a broad range of back-end processing techniques including layer bonding, surface modification and biofunctionalization. This mechanism has been enabled by the excess of epoxide and thiol functional groups on the surface of the OSTE(+) material next to the first and prior to the second curing steps. As a case in point, OSTE(+) materials are able to bond to a broad spectrum of microfluidic materials such as glass, silicon, PMMA, etc without further additional back-end process such as high-temperature thermal bonding, ultrasonic bonding and laser welding, in contrast to pre-dominant microfluidic materials such as glass, PDMS and other polymers which demand such complicated processes. Advantageously, since the thiol-ene polymerization occurs in a benign condition, i.e, lack of harsh solvent use or toxic byproducts, it makes it compatible with protein and cell studies. Moreover, the native excess of thiols on the surface of the OSTE polymer can be utilized for a diverse range of biofunctionalization such as thiol-gold, and also thiol-Michael addition (click) reactions, including thiol-vinyl sulfone, thiol-maleimide, thiol-(meth)acrylate, and thiol-yne interactions. The performed click reaction here provides a robust platform for making layers such as extracellular matrices (ECMs) without any need for implementing additional techniques such as “grafting to” and “grafting from”.

Advantageously, the Micro Reaction Injection Molding (PRIM) technique was implemented to produce OSTE(+) microfluidic devices. Such a technique features several prominent characteristics.

Firstly, it enables batch fabrication of microfluidic devices on a laboratory scale, bridging the gap between single prototyping and industrial production. Here, this concept was realised by producing multiple layers of the microfluidic devices in a single fabrication step. This is in contrast to the predominant technique of PDMS casting which is not scalable.

Secondly, a short cycle time of UV curing process, from 15 seconds up to several minutes based on specific formulations of OSTE(+) resin, is in par with standard injection molding method in the industry. This is in contrast to PDMS prototyping by casting with the cycle time up to hours rendering the process not viable for commercialization of such microfluidic devices. In addition, CNC milling of thermoplastic materials such as PMMA, PC, and PS and micromachining of glass and silicon also suffers from long cycle time (up to hours/days per one prototype, depending on the device complexity).

Thirdly, incorporating both “macro and micro” features in “one injection shot” has been enabled by μRIM. Here, for the first time in OSTE(+) polymers microfluidic devices have been fabricated featuring world-to-chip tube connector interfaces, in macro scale in tandem with micro-channels with the same injection pressure. Such a feature resolves one of the limiting factors in injection molding of microfluidic devices which needs to adapt their injection shot based on the targeted macro/microfeature often implemented by high injection or packing pressure.

Fourthly, here we produce microfluidic devices with high replication fidelity. Whilst the shrinkage in the thermoplastic materials can reach up to 10% or over, our OSTE+ microfluidic devices fabricated via μRIM results in shrinkage less than 1% which in turn offers high replication fidelity. A combination of the delayed gelation phenomenon in thiol-ene polymerization and existence of injection/ventilation ports in the mold design and the resulting polymer backfilling contributes to such a low level of shrinkage.

Method of Using the Microfluidic Device

Also according to the invention there is provided a method of using a microfluidic device as defined above for accommodating, growing, culturing, isolating, treating and/or processing cells, wherein the method comprises the steps of:
(a) adding a cell culture medium into the compartments of the microfluidic device to a level so as to cover and fill the micrometer channel;
(b) adding cells into at least one of the compartments;
(c) connecting the inlet of the first compartment to a pressure control unit; and
(d) modulating the gaseous pressure in the first compartment by the pressure control unit so as to move the cell culture medium between the compartments via the micrometer channel(s).

Preferably, the use of the device is for biological, pharmacological, immunological or toxicological applications.

Conveniently, the cells that are added to the compartment(s) may be selected from the list consisting of primary cells of various tissue origin, for example, primary human hepatocytes, human cancer cell lines; primary murine muscle bundles; and eukaryotic cells.

Advantageously, the cell culture medium does not entirely fill the compartments of the microfluidic device, that is to say, after adding the cell culture medium a gaseous pocket remains in the upper portion of each compartment above the cell culture medium level.

Preferably, cells are not added into the first compartment.

Conveniently, the cells are in the form of 3D tissue models that are generated externally, for instance in ultra-low attachment plates or hanging drops and loaded into the device before the co-culture experiment is to be started.

In the embodiments where the device comprises multiple radial/satellite compartments (e.g., 2, 3, 4, 5, 6, 7, 8, 9, or 10 compartments), preferably each compartment contains a different type of cell, or tissue model.

To mimic physiological inter-tissue communication, the number of 3D units is recommended to be based on the tissues physiological function. For example, adipocyte 3D cultures secrete soluble factors called adipokines, such as adiponectin, and the number of adipocyte 3D cultures are chosen so that the final steady-state concentration of adiponectin in the medium approximates adiponectin concentrations in human plasma (2-20 μg/ml).

Similarly, if pancreatic islets are studied, their number is recommended to be chosen so that insulin levels in the medium upon glucose challenge will resemble physiological postprandial insulin concentrations in the blood (0.5-2 nM).

For example, when loading pancreatic islets into a compartment of the device, 1 to 20 islets may be added, such as 5-10 islets.

Advantageously, in use separate cultures of cells are propagated in each of the radial/satellite compartments and as the gaseous pressure in the first compartment is reduced, cell culture medium is drawn from each of the radial/satellite compartments into the first (central) compartment so that the cell culture medium mixes. On increasing the gaseous pressure in the first compartment the cell culture medium then disperses back out into the radial/satellite compartments and, this way, metabolites and other factors produced by each of the cell cultures can be dispersed to other cell cultures allowing models of bodily systems to be accurately created.

Preferably, the cell culture medium is a DMEM based medium. Advantageously, the DMEM cell culture medium is supplemented with about 2 mM L-glutamine, about 100 units/ml penicillin, about 100 μg/ml streptomycin, about 5.5 μg/ml transferrin, about 6.7 ng/ml sodium selenite, and 100 nM dexamethasone without serum.

The cell culture medium may also comprise carbohydrates, fats and/or insulin depending on the application and the cell line being studied.

Specifically, for studies of glycemic control that involve the culture of liver spheroids, pancreatic islets, adipocyte spheroids and/or skeletal muscle bundles, the cell culture medium may also comprise:
from about 1 mM to about 20 mM glucose concentrations, such as from about 5.5 mM and 11 mM glucose (the lower end of these ranges are known as starvation condition and the upper end is known as fed condition;
from about 0 to about 1000 μM free fatty acids, such as from about 0 to about 480 μM free fatty acids, optionally wherein the free fatty acids are a mixture of oleic and palmitic acid, such as a 1:10 to 10:1 oleic:palmitic acid mixture, for example a 1:1 mixture of oleic and palmitic acid.

Conveniently, the cell culture medium does not comprise any extrinsically added insulin so that insulin levels in the device are internally controlled by the cultured pancreatic islets that respond to high glucose concentration with the release of insulin, whereas low glucose concentrations result in the secretion of glucagon.

Preferably, 3D tissue culture models are loaded to the dedicated compartments in a volume that corresponds to about 10% to about 70% of the total volume of the respective compartment, such as from about 20%-50% of the total volume of the respective compartment.

Advantageously, the central compartment is loaded with a cell culture medium volume that corresponds to about 50 to about 200% of central compartment volume, such as from about 50 to 100% of the central compartment volume. When loading with more than 100% of the internal volume of the first (central) compartment, the cell culture medium flows into the second (radial/satellite) compartments, i.e. the first (central) compartment does not overflow.

In the embodiments where the air-lock element is a liquid air-lock, the liquid air-lock is loaded with oil, or any of the other liquids as defined above, thus linking the tissue model-containing compartments to atmospheric pressure while at the same drastically reducing evaporation and reducing the risk of contamination.

Advantageously, the device then is closed using the upper layer and the upper layer to seal the device. The sealing is implemented through conformal contact of hard layers (PMMA/OSTE) with an elastomeric layer (PDMS) sandwiched in between, through exerting pressure, for example clamping pressure or alternatively using a screw-mounting technique.

Conveniently, the fluidic control unit is connected to the device. Preferably the fluidic control unit is connected via gas-permeable tubing to a programmable high-precision pump that periodically adds and withdraws air to and from the gas phase in the central compartment.

In a preferred embodiment, in use there exists a gas pocket in the top of every compartment. Gas bubbles present in the liquid can thus easily diffuse to such gas compartment. Moreover, PDMS membranes typically close of every compartment, which allows gas to diffuse from the enclosed gas compartment to the surrounding. Such a setup allows control of aspects such as dissolved gasses in the liquid, bubble formation, and water loss via evaporation.

Advantageously, in use the flow between compartments can be modulated to be from about 1 pL/s to about 500 μL/s, such as from about 5 pL/s to about 250 μL/s, for example from about 10 pL/s to about 100 μL/s.

### DETAILED DESCRIPTION OF THE DRAWINGS

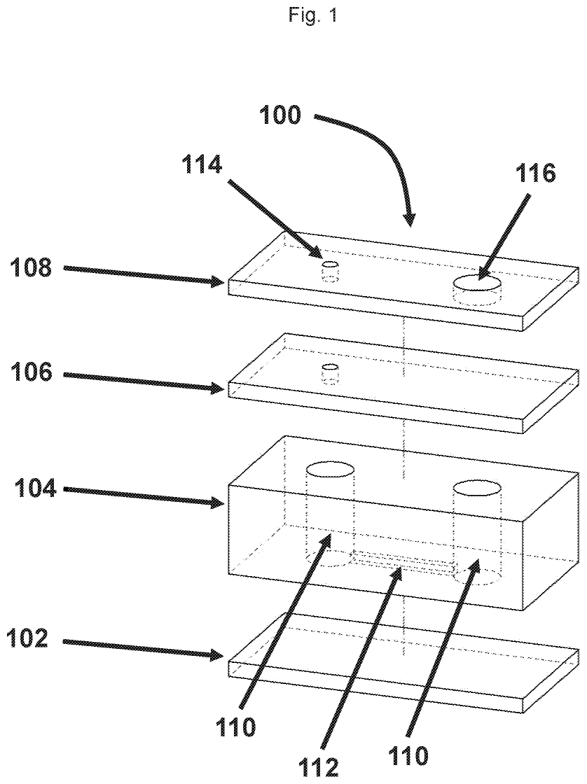

FIG. 1 shows an embodiment of the microfluidic device (100) of the invention (with the air-lock element not shown). The microfluidic device depicted comprises four layers (102, 104, 106, 108) that are positioned together to form a sealed device.

The bottom most layer (102) is a continuous polymer layer made of OSTE or OSTE(+). A second layer (104), which is also referred to above as the “sandwich layer”, is placed so that it lies directly on top of the bottom layer (102). In this embodiment, the second layer (104) is made of PMMA, or alternatively in OSTE (+), although any suitable polymer as outlined above may be used to construct this layer.

The second layer (104) has two cylindrically shaped holes (110) that extend through the plane of the second layer from the upper to the lower layer. The two cylindrically shaped holes are connected via a channel that has been formed in the lower surface of the second layer but does not extend through the entire plane of the second layer.

In this embodiment. the holes have a diameter of about 5 mm and the thickness of the second layer is about 5 mm. The channel has a length of about 20 mm, a width of about 0.3 mm and a depth of about 1 mm.

On being fit together, the exposed and unreacted thiol and/or epoxide groups on the surface of the OSTE/OSTE(+) bottom layer (102) react with the lower surface of the second layer (104) forming covalent bonds between the surface creating a leak proof device without the requirement for any additives, such as an adhesive, that could cause contamination in any future cell culture processes. The bonding is based on the existence of excess epoxide groups on the surface of the polymer after UV curing. The second curing step inside oven triggers the bonding of OSTE layers to other layers.

When placed together, the cylindrical cut outs (110) in the second layer (104) form compartments with the exposed upper side of the bottom layer (102) acting as the bottom of the wells. The total internal volume of each of the wells is about 25 mm³.

A third membrane layer (106) comprised of PDMS is placed on top of the second layer, which allows for gaseous exchange between the internal volume of the compartments and the outside atmosphere.

A final upper layer (108) which is also comprised of PMMA (although any suitable polymer may be used) is then placed on top of the membrane layer (106) to seal the device. The upper layer (108) comprises an inlet (114) positioned directly over one of the compartments, which allows for attachment of a pressure control unit, such as a pump, to allow direct input or retraction of gas into or out of the compartment. The inlet also passes through the third membrane layer (106).

The upper layer (108) also comprises a second aperture (116). This second aperture (116) does not carry on through the third membrane layer (106) and serves to allow gaseous exchange between the compartment it is placed above and the atmosphere external to the device.

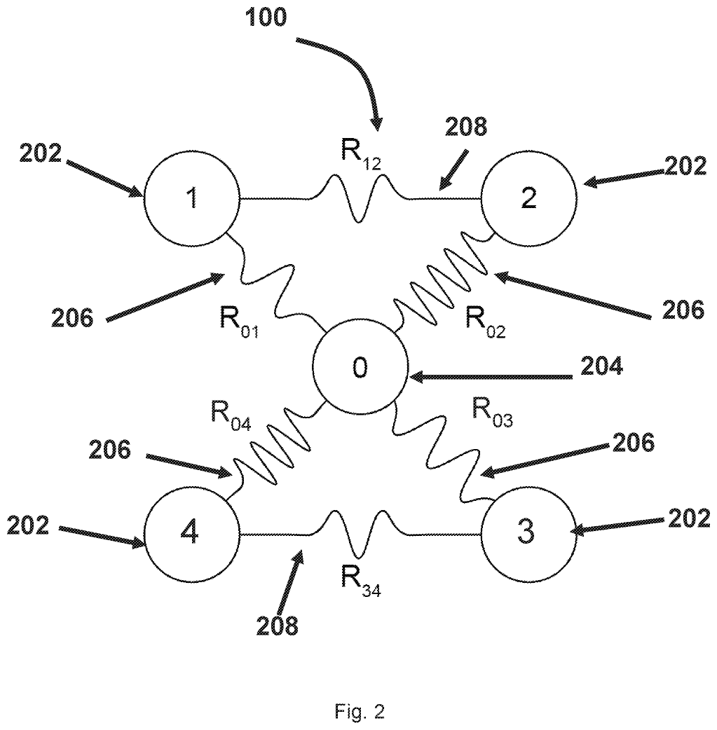

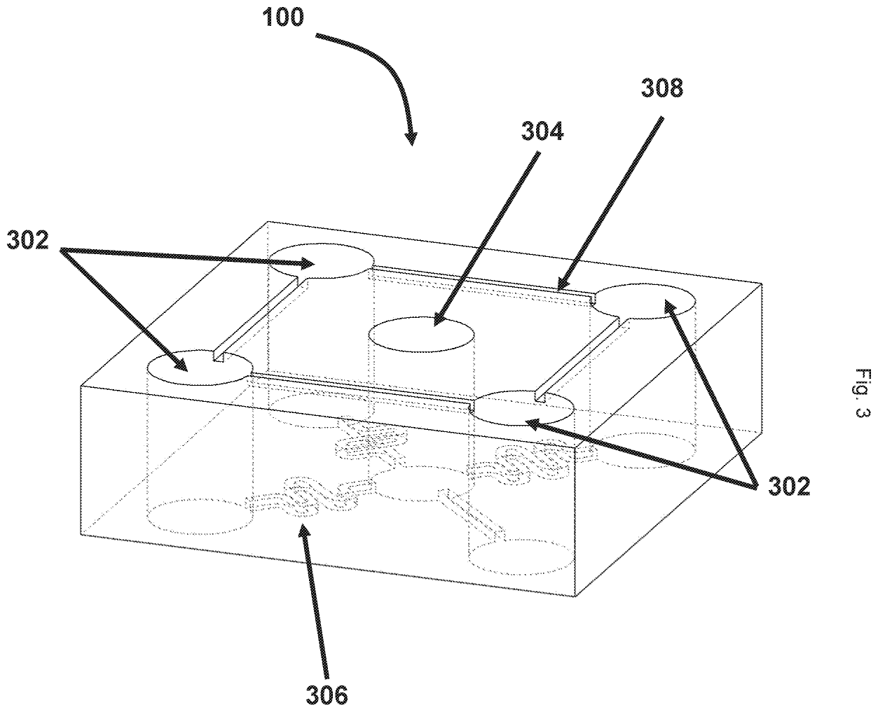

FIG. 2 shows an embodiment of the microfluidic device (100) of the invention (with the air-lock element not shown) comprising five compartments (202, 204) in a top side view (i.e. from the perspective of the user, looking down onto a constructed device).

Four of the compartments (202) are radial (denoted with numbering from 1 to 4) and are all connected to a central compartment (204, denoted as compartment 0) via micrometer channels (206). The inlet for attaching the pressure control unit is not individually depicted in this embodiment, but this is located directly above the central compartment (204).

In this embodiment, the radial compartments are all connected to the central mixing compartment. However, further micrometer channels (208) may be incorporated into the device to connect radial compartments and allow fluid flow therethrough.

All of the micrometer channels in this embodiment are meandering (i.e. have a plurality of bends) which allows for the length of the channels to be altered without repositioning of the radial compartments (202). The meandering channels, and thus the overall length of the channels, modulates fluid flow between the respective compartments. For example, when the air pressure above the liquid level in the central compartment (204) is altered via the pressure control unit, the rate of fluid flow from the radial compartments (202) differs between each compartment depending on the length of the channel. Therefore, when studying interactions between different cell types, the device can be fabricated so as to allow close reproduction of in vivo models.

As an example of how the fluid flow through the device may be controlled, each of the channels may have its own flow rate (denoted Rₓₓ in FIG. 2). If R₀₂+R₁₂<R₀₁, then during every liquid pushing cycle part of well 2 will flow to well 1 without passing central compartment 0 first. During every liquid suction cycle, part of well 1 would flow to well 2 without going to chamber 0.

The main purpose of the microchannels is molecular transport between the compartments. Specifically, the molecular transport allows controlling the concentration of specific molecules in a given compartment. From this perspective, a small flow to a compartment with a low volume has a similar effect as a large flow to a compartment with a large volume.

Advantageously, in use the flow between compartments can be modulated to be from about 1 μL/s to about 500 μL/s, such as from about 5 μL/s to about 250 μL/s, for example from about 10 pL/s to about 100 μL/s.

Although not depicted in this embodiment, the fluid flow rate between compartments may also be altered by varying the internal volume (i.e. the width and/or depth) of the micrometer channels.

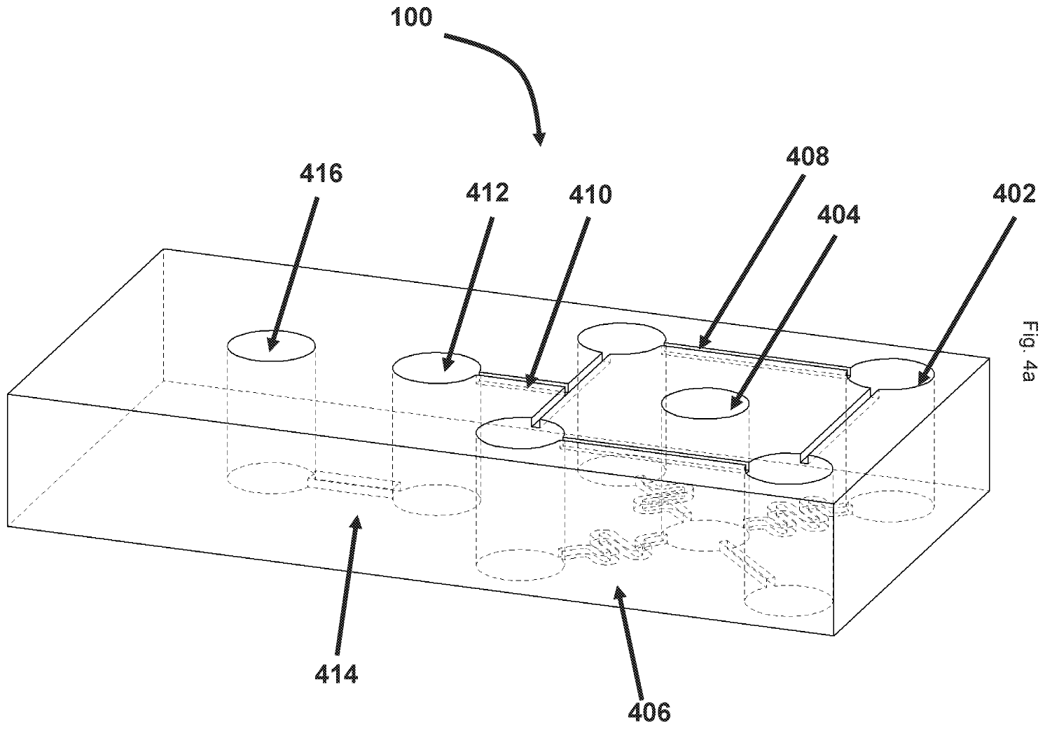

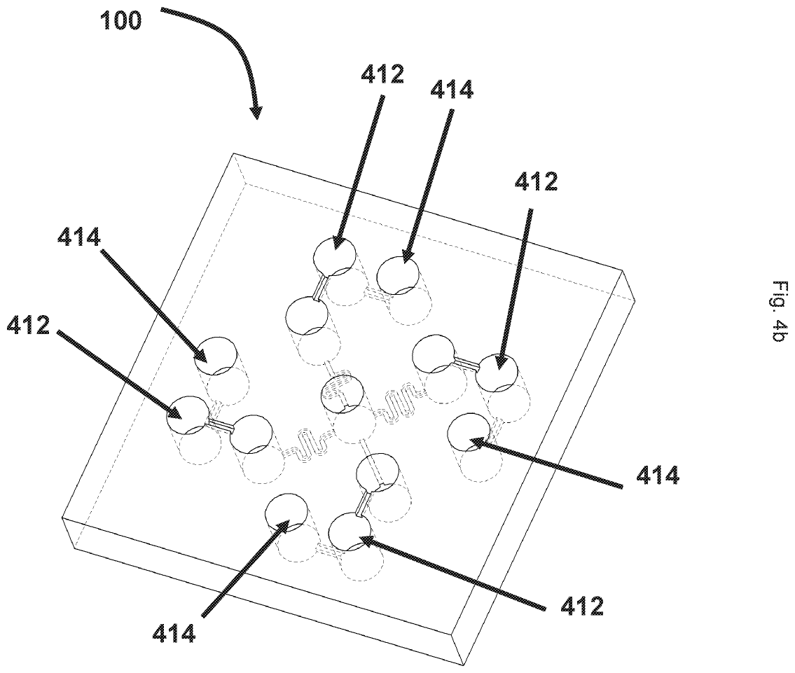

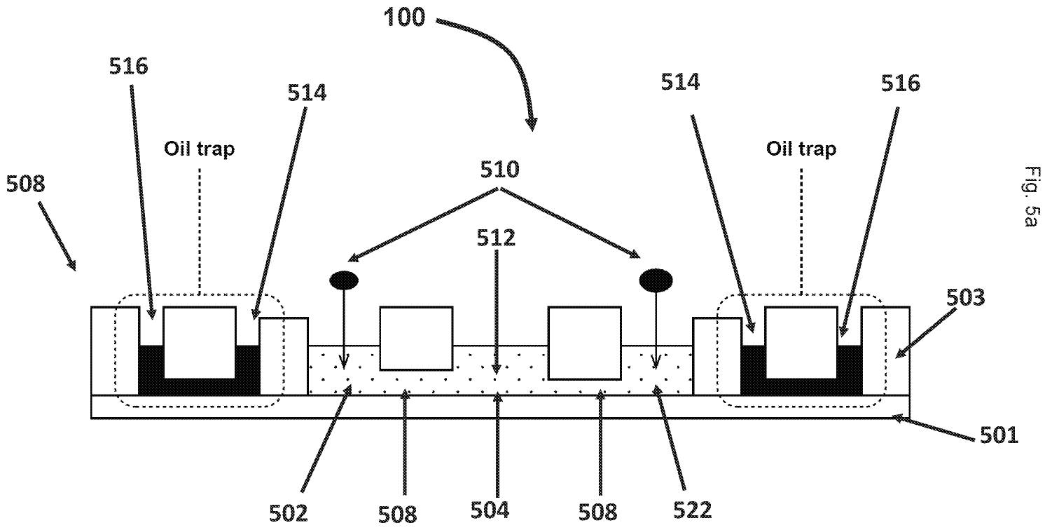

FIGS. 3 and 4 depict two different designs of the device of the invention (100) that both serve the same concept. In these designs, the upper parts of the satellite chambers (302) (i.e. the sections in use that are intended to be the air pockets) are interconnected through upper microchannels (308—only one notated in the figure for clarity) in order to keep the pressure difference across the medium channels (AP) always constant.

FIG. 3 represents the proposed design of the microfluidic device (with the air-lock element not shown).

FIG. 4 illustrates the proposed design of microfluidic device (100) using an air-lock element that is in the form of an oil-lock system. The central compartment (404) is in fluid communication with each of the radial/satellite compartments (402) via individual micrometer channels. The upper section of each of the satellite compartments (402) (i.e. the sections in use that are intended to be the air pockets) are interconnected through upper channels (408—only one notated in the figure for clarity).

A further channel (410) stems from the upper channel connections (408) to the first chamber (412) of the air-lock system. The first chamber (412) of the air-lock system is connected to the second chamber (416) by a lower channel (414) to allow fluid communication therethrough.

In use, the air-lock element of the device of FIG. 4 may be filed with a suitable oil and can be referred to as an “oil-lock. The oil-lock system is devised to maintain the pressure of the air bubbles in satellite compartments at atmospheric pressure while circumventing the reagent evaporation and contamination. The oil-lock system can be designed and fabricated one per satellite compartment (FIG. 4a), or alternatively one for all the satellite compartments (FIG. 4b).

In use, as the air pressure in the upper section of the first compartment (404) increases, this causes fluid in the compartment to flow into the satellite/radial compartments (402). This in turn reduces the available space for the air in the air pocket in the upper section of each of the satellite/radial compartments. Without an air-lock element, this increase in pressure may cause the device to rupture. However, by the interconnecting channels the air can reach the first chamber of the air-lock element.

In use the first and second chambers of the air-lock element may be partly filled with a liquid.

As the air pressure increases in the radial/satellite compartments this moves to the first chamber of the air-lock element and forces the liquid level lower in the first chamber and increases the level in the second chamber (416) as the liquid moves through the lower channel (414).

The second chamber (416) is open to the outside atmosphere either directly, or indirectly via a valve or filter for example. Therefore, this air-lock element allows for the air pressure to equalise in the device without the cell growth medium being exposed directly to the outside atmosphere.

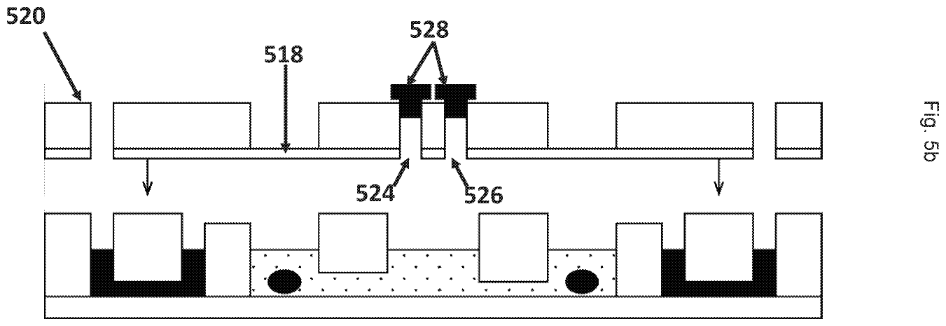

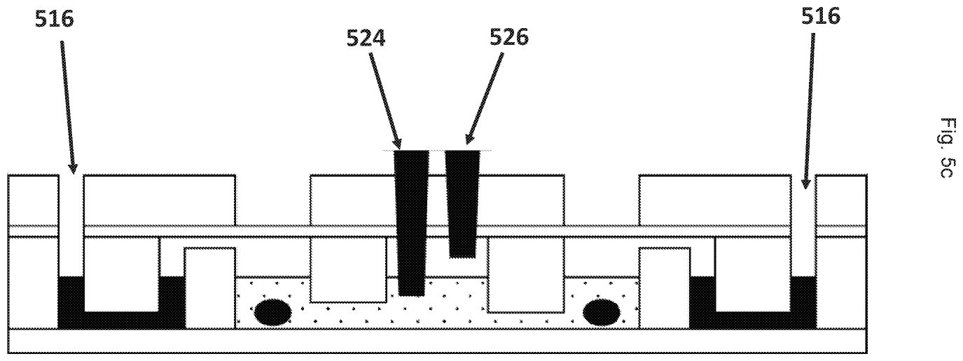

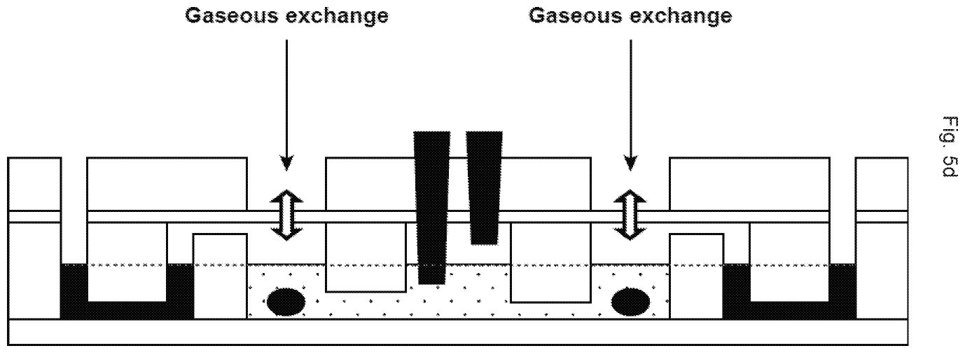

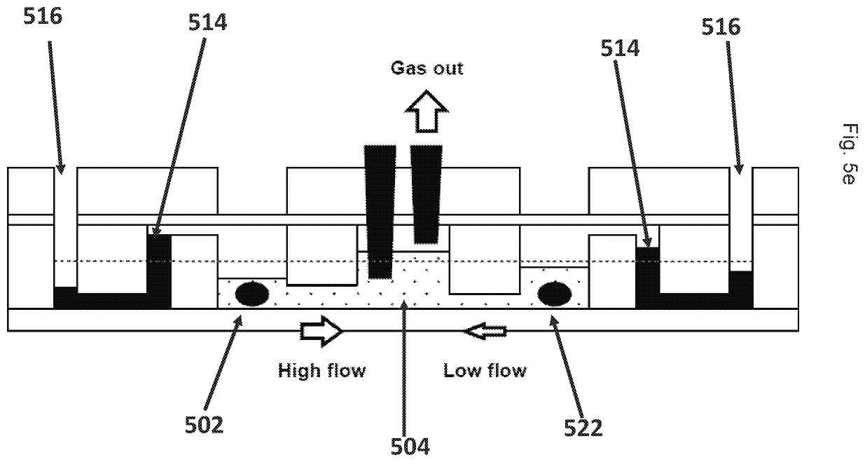

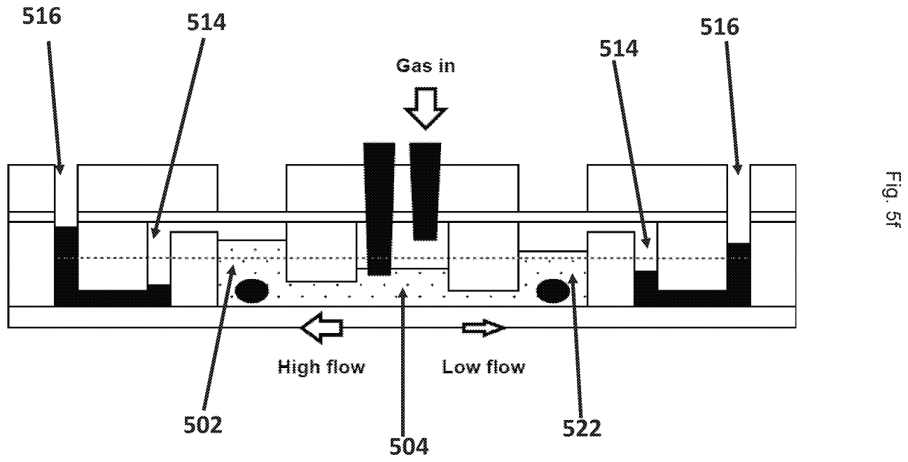

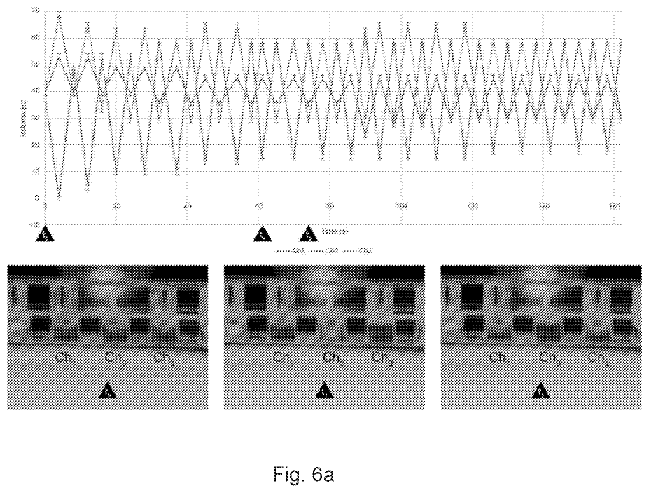

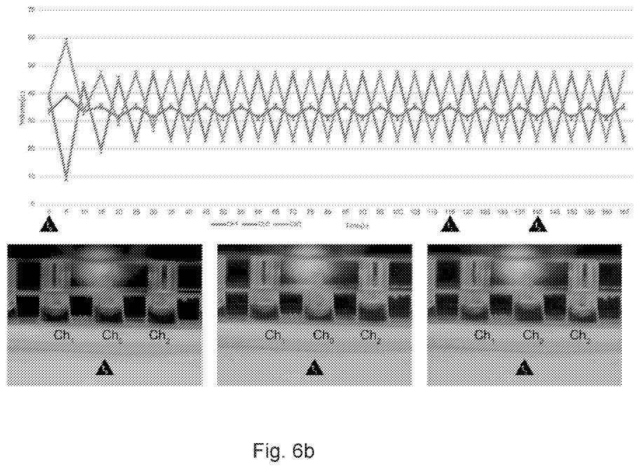

FIGS. 5a to 5f depict an embodiment of the microfluidic device (100) of the invention when in use from a side-view perspective. These figures gradually show from 5a to 5c how the device of this embodiment fits together and how the device is loaded with cells and cell culture medium.

Starting from FIG. 5a, the microfluidic device (100) comprises two satellite compartments (502, 522), each of which are connected to the first chamber (514) of their own individual oil-lock system acting as an air-lock system. Both satellite compartments are connected to a central compartment (504) by micrometer channels (506, 508). Culture medium (512) is introduced into the central compartment (504), which flows into the radial compartments through the micrometer channels. Microtissue samples (510) are then introduced into each of the radial compartments (502, 522). The particular tissue added into the compartments may be the same or different depending on the particular in vivo model being studied. A suitable fluid, such as paraffin oil, other mineral oils or vegetable oils, is then placed into the oil trap.

In this device, the continuous bottom layer (501) and the sandwich layer (503) are composed of OSTE or OSTE(+) polymer. Although depicted as separate layers in this embodiment, they can also be manufactured to be unitary as described above.

Moving to FIG. 5b, after loading the device (100) is then sealed with an upper membrane layer (518) comprised of PDMS. Disposed on top of the layer is a further layer (520) comprised of OSTE. The OSTE/OSTE(+) layer (520) is absent above the radial compartments (502, 522), this is to allow gaseous exchange between the compartments and outer atmosphere.

Two apertures (524, 526) are provided through the two upper layers (518, 520), which are closed with removable stoppers (528). The first aperture (524) is present to allow the user to gain access to the device for procedures such as medium exchange. The second aperture (526) is present as an inlet for connection to a pressure control unit. In FIG. 5c, these two apertures are provided with extensions into the device itself. The extension in the first aperture (524) extends below the liquid line in the device so as to allow for effective medium transfer procedures. The extension in the second aperture (526) extends into the device, but does not pass below the liquid line. This is so as to allow a pressure control unit to be attached to the aperture extension (526) and introduce air into or out of the upper portion of the central compartment so as to flow the fluid through the device.

In this embodiment, the second chamber of the oil-lock element (516) is open to the outside atmosphere.

As can be seen in FIG. 5d, without activating the pressure control unit, the level of liquid in the device is uniform and in this state gaseous exchange through the PDMS membrane layer is able to be achieved.

Under negative pressure actuation (see FIG. 5e), the pressure control unit draws air out of the upper portion of the central compartment (504) pulling liquid into the central compartment. Due to the differences in the geometry of the microchannels connecting the satellite compartments (502, 522) to the central compartment different flow rates through the device are experienced from each of the satellite compartments (502, 522) to central compartment (504). Specifically, the internal volume of the micrometer channel leading from compartment (502) to the central compartment (504) is larger than the volume of the micrometer channel leading from compartment (522) to the central compartment (504). Therefore, the rate of flow from compartment (502) is higher than that from compartment (522).

As can be seen, under negative pressure so as to equalize overall pressure in the device, the oil level in the second chamber (516) of the oil-lock decreases and the corresponding level in the first chamber (514) increases.

Under positive pressure actuation (see FIG. 5f), the pressure control unit pushes air into the upper portion of the central compartment (504) pushing liquid out of the central compartment into the radial compartments (502, 522). Under positive pressure so as to equalize overall pressure in the device, the oil level in the second chamber (516) of the oil-lock increases and the corresponding level in the first chamber (514) decreases.

The device according to the invention may be connected to a pressure control unit that is a syringe pump that is programmed to pump and withdraw frequently. While the syringe connected to the pressure control unit can be either filled with an incompressible fluid (a) or with compressible fluid (b). For each state, the mathematical equations have been derived as below:
a) Syringe filled with an incompressible fluid:
Q_{c}: Central chamber volumetric flow rate
Qₛ: Syringe pump volumetric flow rate
Qₜ: Volumetric flow rate in the i channel
   Hagen-Poiseuille equation:

$$Δ P = \frac{8 μ LQ}{π {R}^{4}}$$

Δp: Pressure difference across a channel,
L: Channel length,
μ: Dynamic viscosity,
Q: Volumetric flow rate
R: Hydraulic radius
ΔP is always constant, therefore only Q and L are variables here:

$$Q = \frac{π {R}^{4} Δ P}{8 μ L} = \frac{α}{L} α = cte$$

Qₛ is known (cte.)
Lᵢ are known
Qᵢ are unknown

$${Q}_{s} = {Q}_{1} + {Q}_{2} + {Q}_{3} + {Q}_{4} \\ {Q}_{s} = \frac{α}{{L}_{1}} + \frac{α}{\frac{{L}_{1} ( {L}_{2}}{L} {}_{1} )} + \frac{α}{{L}_{1} ( {L}_{3} / {L}_{1} )} + \frac{α}{\frac{{L}_{1} ( {L}_{4}}{L} {}_{1} )} \\ {Q}_{s} = {Q}_{1} + \frac{{Q}_{1}}{\frac{{L}_{2}}{{L}_{1}}} + \frac{{Q}_{1}}{\frac{{L}_{3}}{{L}_{1}}} + \frac{{Q}_{1}}{\frac{{L}_{4}}{{L}_{1}}} = {L}_{1} {Q}_{1} ( \frac{1}{{L}_{1}} + \frac{1}{{L}_{2}} + \frac{1}{{L}_{3}} + \frac{1}{{L}_{4}} ) \to {Q}_{1} = \frac{{Q}_{s}}{{L}_{1} ( \frac{1}{{L}_{1}} + \frac{1}{{L}_{2}} + \frac{1}{{L}_{3}} + \frac{1}{{L}_{4}} )} \\ {Q}_{2} = \frac{{Q}_{1}}{{L}_{2} / {L}_{1}} , {Q}_{3} = \frac{{Q}_{1}}{{L}_{3} / {L}_{1}} , {Q}_{4} = \frac{{Q}_{1}}{{L}_{4} / {L}_{1}}$$

The equivalent circuit model also leads to the same result:

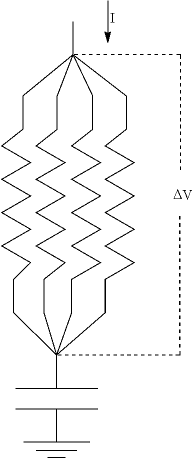

$$I = {I}_{1} + {I}_{2} + {I}_{3} + {I}_{4} = Σ {I}_{i} \\ Δ V = IR = cte \\ \frac{1}{R} = \frac{1}{{R}_{1}} + \frac{1}{{R}_{2}} + \frac{1}{{R}_{3}} + \frac{1}{{R}_{4}} = Σ \frac{1}{{R}_{i}} \\ {I}_{1} = \frac{Δ V}{{R}_{1}} = \frac{IR}{{R}_{1}} = \frac{I}{{R}_{1} Σ \frac{1}{{R}_{i}}} \\ {I}_{j} = \frac{I}{{R}_{j} {Σ}_{1}^{i} \frac{1}{{R}_{i}} \to {Q}_{j} = \frac{{Q}_{s}}{{L}_{j} {Σ}_{1}^{i} \frac{1}{{L}_{i}}}}$$

b) Syringe filled with a compressible fluid (air)
Here instead of dealing with instantaneous rates, the average rates (Q̅ᵢ) during a half-cycle (for example the compression step) can be derived as below:

Temperature=cte.=>P₀V₀=PₜVₜ
t=Time at the end of the compression step (half-cycle time)
Qₛ=Syringe pump's volumetric flow rate
Q̅_{c}=Average flow rate from central chamber to satellite chambers
Vₛ=Gas volume inside syringe pump, tubing and central chamber
Vₐ=Gas volume inside satellite chambers (air bubbles) and the interconnecting microchannels
Pₛ_{ₜ}=Syringe pump pressure at time t
Pₐ_{ₜ}=Air bubble's pressure at time t
System of linear equations:

$$\{ \begin{matrix}{P}_{Atm} {V}_{s} = {P}_{{s}_{t}} ( {V}_{s} - {Q}_{s} t + {\overline{Q}}_{c} t ) \\ {P}_{Atm} {V}_{a} = {P}_{{a}_{t}} ( {V}_{a} - {\overline{Q}}_{c} t )\end{matrix} \}$$

At the half-cycle time when the flow rates are zero Pₛ_{ₜ}=Pₐ_{ₜ}

$${P}_{Atm} {V}_{s} = \frac{{P}_{Atm} {V}_{a}}{{V}_{a} - {\overline{Q}}_{c} t} ( {V}_{s} - {Q}_{s} t + {\overline{Q}}_{c} t ) \to we can find {\overline{Q}}_{c} \\ Q = \frac{π {R}^{4} Δ P}{8 μ L} = \frac{α}{L} α = equal for all branches at every moment \\ {\overline{Q}}_{c} = {\overline{Q}}_{1} + {\overline{Q}}_{2} + {\overline{Q}}_{3} + {\overline{Q}}_{4} \\ {\overline{Q}}_{c} = {\overline{Q}}_{1} + \frac{{\overline{Q}}_{1}}{\frac{{L}_{2}}{{L}_{1}}} + \frac{{\overline{Q}}_{1}}{\frac{{L}_{3}}{{L}_{1}}} + \frac{{\overline{Q}}_{1}}{\frac{{L}_{4}}{{L}_{1}}} = {L}_{1} {\overline{Q}}_{1} ( \frac{1}{{L}_{1}} + \frac{1}{{L}_{2}} + \frac{1}{{L}_{3}} + \frac{1}{{L}_{4}} ) \to {\overline{Q}}_{1} = \frac{{\overline{Q}}_{c}}{{L}_{1} ( \frac{1}{{L}_{1}} + \frac{1}{{L}_{2}} + \frac{1}{{L}_{3}} + \frac{1}{{L}_{4}} )} \\ {\overline{Q}}_{2} = \frac{{\overline{Q}}_{1}}{{L}_{2} / {L}_{1}} , {\overline{Q}}_{3} = \frac{{\overline{Q}}_{1}}{{L}_{3} / {L}_{1}} , {\overline{Q}}_{4} = \frac{{\overline{Q}}_{1}}{{L}_{4} / {L}_{1}}$$

### EXAMPLES

The present invention will be further described by reference to the following examples which are not intended to limit the scope of the invention.

### Example 1—Method of Making the Device

Below is a general description of how the device depicted in FIG. 5 can be manufactured according to the invention.

The layers of the device composed of OSTE(+) were prepared by injection of OSTEO) resin (OSTEMER 322, Mercene Labs, Sweden) inside CNC processed molds preferably made of aluminium. Depending on the targeted layer, the mold can either contain a cavity to produce a flat layer or alternatively micromachined with more detailed features such as micro-channels and ports intended for tube connectors. UV curing of assembled mold. This was followed by demolding of replica out of mold, either manually or by ejector pins.

The membrane layer was fabricated via the casting of PDMS ((Sylgard 184) resin inside a cavity (with a thickness of 100-1000 μm) and subsequent thermal curing from 30 to 60 minutes depending on the layer thickness. This resulted in a flat PDMS layer.

The sandwich layer was either directly micromachined for the purpose of flexible prototyping or alternatively μRIMed when the optimized feature sizes are established. The μRIM of the sandwich layer follows the steps as stated above.

Ultimately, all layers were assembled together and underwent a second the curing step via exposure to heat in an oven with a temperature of 80° C. for 120 minutes. This led to strong covalent bonding of all layers together expect for PDMS layer relies on conformal contact. The subsequent use of clamps on screws will exert more pressure leading to a robust and leak-free microfluidic device.

### Example 2—Controlling Flow Through the Device

FIGS. 6A and 6B show the measurement results of two microfluidic devices, based on the oil-lock (liquid air-lock) concept, comprised of three main chambers: Central chamber (Ch₀), satellite chamber connected to the central chamber through a meander channel (Ch₁), satellite chamber connected to the central chamber through a straight channel (Ch₂). The length of the meander channels are two and five times longer than those of straight channels for FIGS. 6A and 6B, respectively. The associated measurement graphs precisely demonstrate how the liquid volume related to the three chambers proportionally change in a controllable way during a certain time period while the syringe pump is programmed to pump and withdraw frequently (at the syringe flow rate of 3 ml/min). For each graph, three snapshots of the device at different time points (t₀, t₁, t₂) are depicted.

### Example 3—Cell Stability Studies

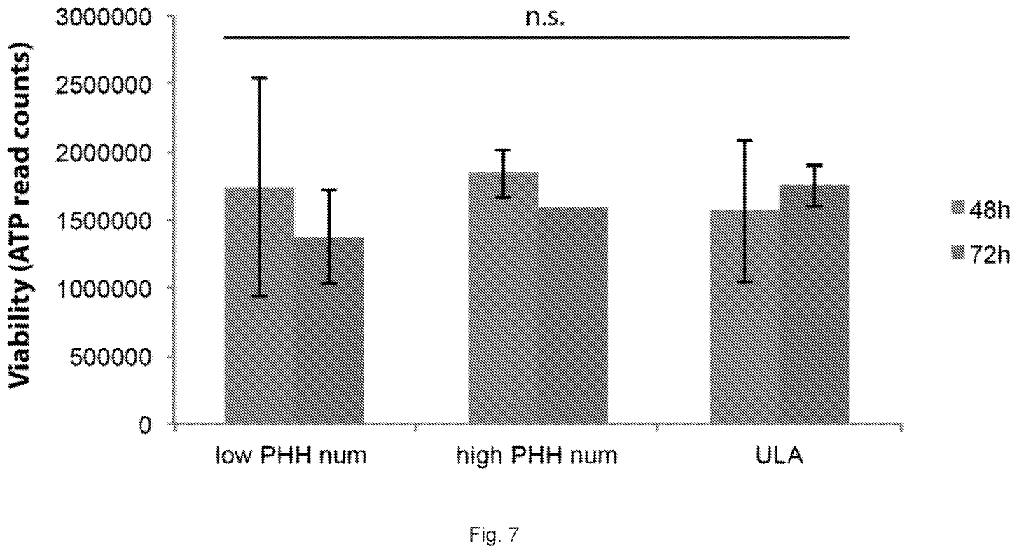

The long-term stability of primary human cells in the device according to the microfluidic device as depicted in FIG. 7 was studied and compared to a standard ULA as reported in Bell et al. Sci Rep 2016.

The results show that the primary human liver cells are as viable as the standard at 48 and 72 hour time points.

### Example 4—Use of the Device for Biological, Pharmacological, Immunological or Toxicological Applications

General Cell Growing Protocol

3D model tissues were generated externally in ultra-low attachment plates or hanging drops and then loaded into one dedicated compartment of the device. The number of 3D units were based on the tissues physiological function.

A cell culture medium was added into the compartments of the microfluidic device to a level so as to cover and fill the micrometer channel, but not fill the compartments and leave a gaseous pocket in at least the first compartment. A DMEM base medium was used as a culture medium and supplemented with 2 mM L-glutamine, 100 units/ml penicillin, 100 μg/ml streptomycin, 5.5 μg/ml transferrin, 6.7 ng/ml sodium selenite, 100 nM dexamethasone without serum and custom concentrations of carbohydrates, fats and insulin as needed.

3D tissue culture models were loaded to the dedicated compartments in a volume that corresponded to 20%-50% of the total volume of the respective compartment. The central compartment was loaded with a medium volume that corresponded to 80% of central compartment volume. The liquid air-lock system was loaded with oil to link the tissue model-containing compartments to atmospheric pressure and to reducing evaporation and the risk of contamination.

Subsequently, the device was closed and sealed by clamping the upper layer to the middle layer. The pneumatic actuator outlet was connected via gas-permeable tubing to a programmable high-precision pump that periodically added and withdrew air to and from the gas phase in the central compartment.

Adipocyte 3D Cultures

The general protocol was followed. The number of adipocyte 3D cultures were chosen so that the final steady-state concentration of adiponectin in the medium approximated adiponectin concentrates in human (2-20 μg/ml).

Pancreatic Islets for Studies of Glycemic Control

The General protocol was followed. The number of pancreatic islets were chosen so that insulin levels in the medium upon glucose challenge will resemble physiological postprandial insulin concentrations in the blood (0.5-2 nM).

The use of 5-10 islets was sufficient to reach these concentrations in a culture medium volume of 100 μl within 10-30 minutes.

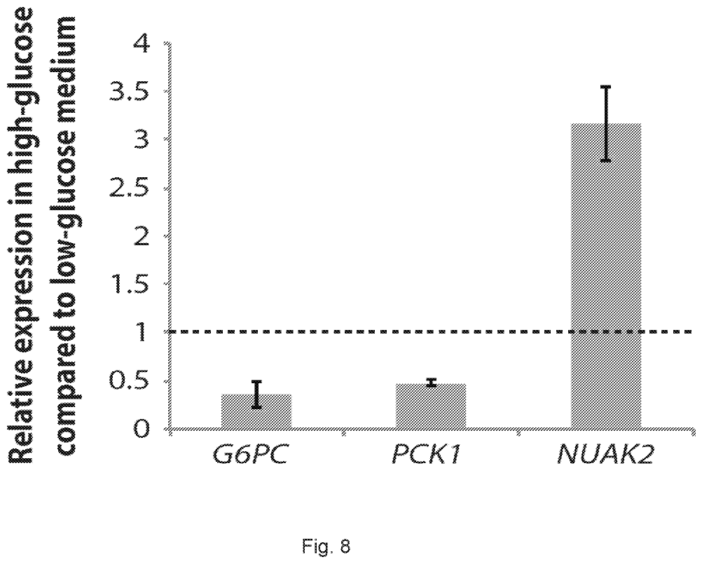

The glucose concentrations were kept between 5.5 mM (starvation conditions) and 11 mM glucose (fed conditions). Free fatty acids (1:1 mixtures of oleic and palmitic acid) were kept at a concentration of 0-480 μM. No insulin was extrinsically added in order to keep insulin levels in the device controlled internally by the cultured pancreatic islets. The cultured pancreatic islets responded to high glucose concentration with the release of insulin and to a low glucose concentrations with the secretion of glucagon. In co-culture chips with human liver spheroids and pancreatic islets, insulin responsive genes in the liver compartment (Batista et al. Cell Reports 2019) changed in expression levels as a function of glucose concentrations. Specifically, high glucose levels resulted in downregulation of the gluconeogenesis genes G6PC and PCK1, known to be repressed by insulin, whereas the insulin-induced gene NUAK2 was increased (FIG. 8).

1. A microfluidic device comprising:
a first compartment comprising an inlet that is connectable to a fluidic control unit;
a second compartment;
a micrometer channel connecting the first and second compartments so as to allow fluid communication between the first and second compartments; and
an air-lock element in fluid communication with the second compartment, wherein the air-lock element is configured so that in use the internal atmosphere of the device is sealed from the external atmosphere and so that when fluid is introduced or withdrawn from the first compartment via the inlet the air-lock element maintains an overall constant pressure within the device.

2. The microfluidic device of claim 1, wherein the fluidic control unit is a gaseous fluid control unit.

3. The microfluidic device of claim 1, wherein the air-lock element comprises a first chamber and a second chamber connected via a lower channel to allow fluid communication therethrough and wherein the first chamber is connected to the second compartment, either directly or indirectly, via an upper channel to allow fluid communication therethrough.

4. The microfluidic device of claim 3, wherein the second chamber of the air-lock element comprises an outlet to allow fluid flow into and out of the second chamber.

5. The microfluidic device of claim 4, wherein the second chamber comprises a base, wherein the outlet is positioned in the device at a location further from the base of the second chamber than the lower channel.

6. The microfluidic device of claim 1, wherein the first compartment comprises a base, wherein the inlet is positioned in the device at a location further from the base of the first compartment than the micrometer channel.

7. The microfluidic device of claim 1, wherein the microfluidic device comprises a further third compartment in fluid communication with the first compartment via a micrometer channel.

8. The microfluidic device of claim 1, wherein the microfluidic device comprises multiple further compartments, each of which are in fluid communication with the first compartment via micrometer channels.

9. The microfluidic device of claim 1, wherein the micrometer channel(s) has/have, independently, a length of from about 0.1 to about 100 mm.

10. The microfluidic device of claim 1, wherein the micrometer channel(s) have a hydraulic diameter, independently, of from about 1 to about 2000 μm.

11. The microfluidic device of claim 1, wherein the micrometer channel(s) is/are, independently, essentially straight or tortuous.

12. The microfluidic device of claim 1, wherein the internal walls of the compartments and the micrometer channels and/or the floor surface of the compartments are made of a polymer selected from the list of poly(dimethyl siloxane) (PDMS); a thiol-ene polymer; a polymer with thiol, ene and/or epoxide groups on the surface; cyclic olefin copolymer (COC); polystyrene (PS); polycarbonate (PC); or an acrylic.

13. The microfluidic device according to claim 1, wherein the microfluidic device is comprised of:
a) a bottom continuous layer;
b) an upper layer; and
c) a sandwich layer disposed between the bottom continuous layer and the upper layer, wherein the sandwich layer comprises at least two cut-outs extending through the plane of the sandwich layer and a micrometer channel connecting the two cut-outs, wherein the at least two cut-outs and the surface of the bottom continuous layer in contact with the sandwich layer define the first and second compartments.

14. The microfluidic device of claim 1, wherein the microfluidic device further comprises at least one gas permeable membrane in connection with at least one of the compartments.

15. The microfluidic device according to claim 13, wherein the bottom continuous layer is comprised of the same material as the sandwich and/or upper layer, or wherein the bottom continuous layer is comprised of a polymer with thiol and/epoxide groups on the surface of the layer disposed towards the sandwich layer.

16. The microfluidic device according to claim 13, wherein the upper layer and, if present, the membrane layer comprise an aperture extending therethrough and positioned above one of the compartments so as to define the inlet.

17. The microfluidic device of claim 1, wherein one or more of the second, third or other compartments comprise one or more capillary valves positioned in the compartment to allow connection between the cell culture medium in use and the external atmosphere.

18. A method of using a microfluidic device according to claim 1 for accommodating, growing, culturing, isolating, treating and/or processing cells, wherein the method comprises the steps of:
(a) adding a cell culture medium into the compartments of the microfluidic device to a level so as to cover and fill the micrometer channel;
(b) adding cells into at least one of the compartments;
(c) connecting the inlet of the first compartment to a pressure control unit; and
(d) modulating the gaseous pressure in the first compartment by the pressure control unit so as to move the cell culture medium between the compartments via the micrometer channel.

19. A kit-of-parts comprising a microfluidic device according to claim 1 in the form of a sterile, pre-packaged kit-of-parts for single use.
